(19) United States
(12) Patent Application Publication
(10) Pub. No.: US 2024/0225657 A1
(43) Pub. Date: Jul. 11, 2024

(54) LEFT ATRIAL APPENDAGE CLOSURE DEVICE WITH INTEGRATED FEATURES ENCOURAGING TISSUE INGROWTH

(71) Applicant: BOSTON SCIENTIFIC SCIMED, INC., MAPLE GROVE, NM (US)

(72) Inventors: Carly Rogers, Eden Prairie, MN (US); Nicholas Lee Tassoni, Andover, MN (US); David John Onushko, Maple Grove, MN (US); Richard Budnar, Chaska, MN (US)

(73) Assignee: BOSTON SCIENTIFIC SCIMED, INC., MAPLE GROVE, MN (US)

(21) Appl. No.: 18/406,765

(22) Filed: Jan. 8, 2024

Related U.S. Application Data
(60) Provisional application No. 63/437,808, filed on Jan. 9, 2023.

(51) Int. Cl. A61B 17/12

(52) U.S. Cl. CPC A61B 17/12122; A61B 17/1215; A61B 17/12195; A61B 2017/1205

(57) ABSTRACT

An implantable medical device such as a left atrial appendage closure (LAAC) device includes an expandable frame that is moveable between a collapsed configuration for delivery and an expanded configuration for deployment. One or more space filling elements that are adapted to facilitate thrombus formation are secured to the expandable frame. An expandable element spans at least part of the expandable frame.

### CROSS-REFERENCE TO RELATED APPLICATIONS

This application claims the benefit of U.S. Provisional Patent Application Ser. No. 63/437,808, filed Jan. 9, 2023, which is incorporated herein by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device. The LAAC device includes an expandable frame moveable between a collapsed configuration for delivery and an expanded configuration for deployment, one or more space filling elements constrained by the expandable frame, and an occlusive element spanning at least part of the expandable frame. The one or more space filling elements are adapted to facilitate thrombus formation within the LAAC device.

Alternatively or additionally, at least some of the one or more space filling elements may be constrained by being secured to the expandable frame prior to implantation of the LAAC device.

Alternatively or additionally, at least some of the one or more space filling elements may be welded to the expandable frame.

Alternatively or additionally, at least some of the one or more space filling elements may be adhesively secured to the expandable frame.

Alternatively or additionally, the expandable frame may further include a proximal hub and a distal hub, and at least some of the one or more space filling elements may be secured to at least one of the proximal hub and the distal hub.

Alternatively or additionally, at least some of the one or more space filling elements may be constrained by being trapped within the expandable frame prior to implantation.

Alternatively or additionally, at least some of the one or more space filling elements may include an embolic coil.

Alternatively or additionally, at least some of the one or more space filling elements may include fibrous material.

Alternatively or additionally, the expandable frame may include a plurality of struts forming a plurality of strut intersections, and at least some of the one or more space filling elements may include fibers each extending between two or more of the plurality of struts and/or two or more of the plurality of strut intersections.

Alternatively or additionally, at least one of the one or more space filling elements include an expandable element that is adapted to be secured to the expandable frame and one or more fibers extending from the expandable element.

Alternatively or additionally, the expandable frame may include a first expandable feature that is moveable between a collapsed configuration for delivery and an expanded configuration for deployment, and a second expandable feature that is secured relative to the first expandable feature and that is moveable between a collapsed configuration for delivery and an expanded configuration for deployment.

Alternatively or additionally, at least some of the one or more space filling elements may extend between the first expandable feature and the second expandable feature.

Alternatively or additionally, at least some of the one or more space filling elements may be disposed within at least one of the first expandable feature and the second expandable feature.

Alternatively or additionally, the LAAC device may further include a coating disposed over at least a portion of the LAAC device, the coating adapted to encourage thrombus formation.

Another example may be found in a left atrial appendage closure (LAAC) device. The LAAC device includes an expandable frame moveable between a collapsed configuration for delivery and an expanded configuration for deployment, one or more expandable elements that are secured to the expandable frame and are adapted to facilitate thrombus formation within the LAAC device, and an occlusive element spanning at least part of the expandable frame.

Alternatively or additionally, at least some of the one or more expandable elements may include an embolic coil.

Alternatively or additionally, at least some of the one or more expandable elements may be secured to the expandable frame prior to implantation.

Alternatively or additionally, at least some of the one or more expandable elements may be adapted to move between a collapsed configuration for delivery and an expanded configuration for deployment.

Another example may be found in a left atrial appendage closure (LAAC) device. The LAAC device includes an expandable frame moveable between a collapsed configuration for delivery and an expanded configuration for deployment, one or more fibers secured relative to the expandable frame, the one or more fibers adapted to facilitate thrombus formation within the LAAC device, and an occlusive element spanning at least part of the expandable frame.

Alternatively or additionally, the LAAC device may further include a support element extending to a position exterior of the expandable frame, and the one or more fibers may be secured to the support element at the position exterior of the expandable frame and extend back into the expandable frame.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional view of an LAA (left atrial appendage);

FIG. 2 is a perspective view of an illustrative LAAC (left atrial appendage closure) device, shown without a covering;

FIG. 3 is a perspective view of the illustrative LAAC device of FIG. 2, including a covering;

FIG. 4 is a perspective view of an illustrative LAAC device;

FIG. 4A is an enlarged view of a portion of the illustrative LAAC device of FIG. 4;

FIG. 4B is a schematic cross-section of a portion of the illustrative LAAC device of FIG. 4, such as a portion of the expandable frame and/or a portion of a space filling element;

FIG. 5 is a schematic view of an illustrative LAAC device;

FIG. 6 is a schematic view of an illustrative LAAC device;

FIG. 7 is a schematic view of an illustrative LAAC device;

FIG. 8 is a schematic view of an illustrative LAAC device;

FIG. 9 is a schematic view of an illustrative LAAC device;

FIG. 10 is a schematic view of an illustrative LAAC device;

FIG. 11 is a side view of an illustrative LAAC device;

FIG. 12 is a side view of an illustrative LAAC device;

FIG. 13 is a schematic view of an illustrative LAAC device;

FIG. 14 is a schematic view of an illustrative LAAC device;

FIG. 15 is a schematic view of an illustrative LAAC device including a first expandable feature and a second expandable feature;

FIG. 16 is a schematic view of an illustrative LAAC device including a first expandable feature and a second expandable feature; and

FIG. 17 is a schematic view of an illustrative LAAC device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result).

In many instances, the terms “about” mayinclude numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” maybe arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

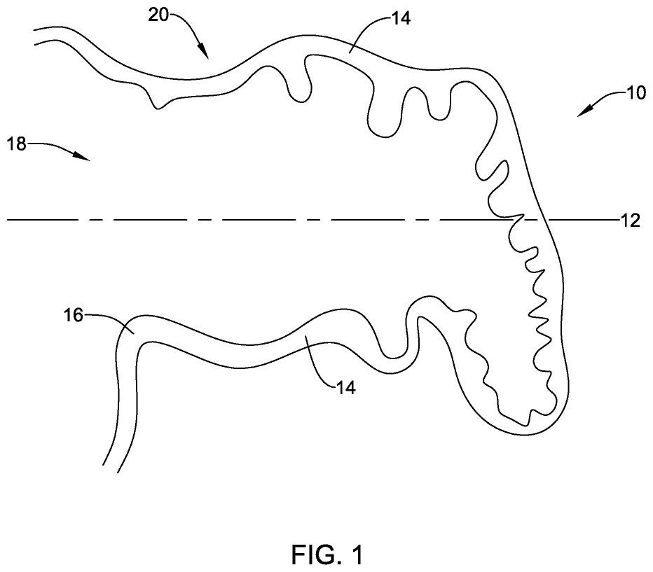

FIG. 1 is a partial cross-sectional view of a left atrial appendage 10. In some embodiments, the left atrial appendage (LAA) 10 mayhave a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 mayinclude a generally longitudinal axis 12 arranged along a depth of a main body 20 of the left atrial appendage 10. The main body 20 mayinclude a lateral wall 14 and an ostium 16 forming a proximal mouth 18. In some examples, a lateral extent of the ostium 16 and/or the lateral wall 14 maybe smaller or less than a depth of the main body 20 along the longitudinal axis 12, or a depth of the main body 20 maybe greater than a lateral extent of the ostium 16 and/or the lateral wall 14. In some examples, the LAA 10 maynarrow quickly along the depth of the main body 20 or the left atrial appendage may maintain a generally constant lateral extent along a majority of depth of the main body 20. In some examples, the LAA 10 mayinclude a distalmost region formed or arranged as a tail-like element associated with a distal portion of the main body 20. In some examples, the distalmost region may protrude radially or laterally away from the longitudinal axis 12.

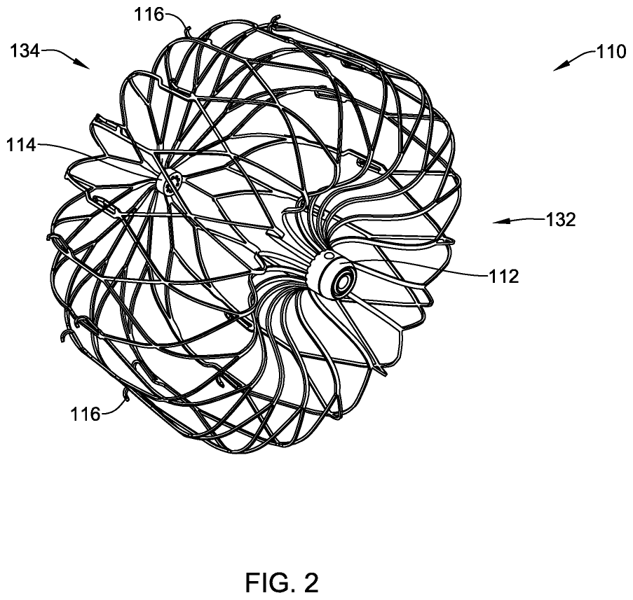

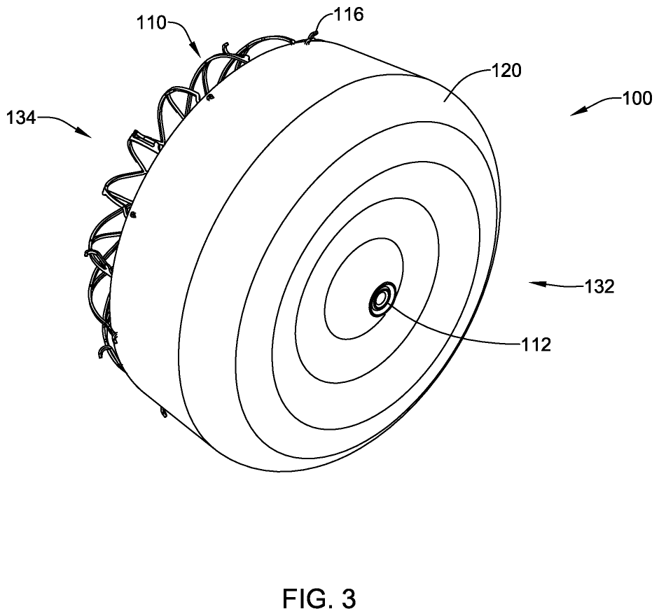

In some instances, a device known as an LAAC (left atrial appendage closure) device may be implanted within the LAA 10, such as near or within the ostium 16, in order to seal off the interior of the LAA 10 from the rest of the heart interior. FIGS. 2 and 3 provide views of a left atrial appendage closure (LAAC) device 100. The LAAC device 100 may include an expandable frame 110 configured to shift axially and/or radially along a central longitudinal axis between the fully constrained configuration and the fully unconstrained configuration. In the fully constrained configuration, the expandable frame 110 may be axially elongated and/or radially compressed. In the fully unconstrained configuration, the expandable frame 110 may be axially shortened and/or radially expanded.

As seen in FIG. 3, which illustrates selected features of the LAAC device 100 in the fully unconstrained configuration, the expandable frame 110 may have a plurality of struts disposed about the central longitudinal axis. In some embodiments, the plurality of struts may define a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements.

The expandable frame 110 may include a proximal hub 112 and a distal hub 114. The expandable frame 110 may be considered as having a proximal-facing side 132 and a distally-facing side 134. It will be appreciated that the proximal-facing side 132 will face proximally and the distal-facing side 134 will face distally when the LAAC device 100 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 100 and/or the expandable frame 110 to a delivery device. In some embodiments, the proximal hub 112 may include internal threads configured to rotatably and/or threadably engage an externally threaded distal end of a delivery device. Other configurations for releasably securing the LAAC device 100 to a delivery device are also contemplated. As noted herein, some features are not shown in every figure to improve clarity.

The expandable frame 110 and/or the plurality of struts may be formed and/or cut from a tubular member. In some embodiments, the expandable frame 110 and/or the plurality of struts may be integrally formed and/or cut from a unitary member. In some embodiments, the expandable frame 110 and/or the plurality of struts may be integrally formed and/or cut from a unitary tubular member and subsequently formed and/or heat set to a desired shape in the fully unconstrained configuration. In some embodiments, the expandable frame 110 and/or the plurality of struts may be integrally formed and/or cut from a unitary flat member or sheet, and then rolled or formed into a tubular structure and subsequently formed and/or heat set to the desired shape in the fully unconstrained configuration. Some exemplary means and/or methods of making and/or forming the expandable frame 110 and/or the plurality of struts include laser cutting, machining, punching, stamping, electro discharge machining (EDM), chemical dissolution, etc. Other means and/or methods are also contemplated.

As would be understood by the skilled person, anatomical features may vary in size and/or shape. In some embodiments, the left atrial appendage may have an irregular (e.g., elongated and/or oblong) cross-sectional shape. In some embodiments, the expandable frame 110 may be compliant and substantially conform to and/or be in sealing engagement with the shape and/or geometry of a lateral wall of a left atrial appendage when deployed and/or expanded therein. In some embodiments, the left atrial appendage closure device 100 may expand to a size, extent, or shape less than or different from the fully unconstrained configuration, as determined by the surrounding tissue and/or lateral wall of the left atrial appendage. In some embodiments, the expandable frame 110 may be configured to shape and/or stretch the tissue of the left atrial appendage such that the lateral wall of the left atrial appendage substantially conforms to an outer shape of the expandable frame 110. Other configurations are also contemplated.

In some embodiments, the expandable frame 110 may include at least one anchoring member 116 extending radially outward therefrom in the fully unconstrained configuration. In some embodiments, the expandable frame 110 may include at least one anchoring member 116 extending radially outward from the expandable frame 110. In some embodiments, the expandable frame 110 may include at least one anchoring member 116 extending radially outward from the expandable frame 110 near a proximal shoulder of the expandable frame 110. In some embodiments, the expandable frame 110 may include at least one anchoring member 116 extending radially outward from the expandable frame 110 proximate a midsection of the expandable frame 110. In some embodiments, the at least one anchoring member 116 may be configured to engage with the lateral wall of the main body of the left atrial appendage. In some embodiments, the at least one anchoring member 116 may be formed as J-shaped hooks having a free end extending in and/or directed toward a proximal direction with respect to the central longitudinal axis of the left atrial appendage closure device 100 and/or the expandable frame 110. Other configurations are also contemplated.

In some embodiments, the left atrial appendage closure device 100 may optionally include an occlusive element 120 connected to, disposed on, disposed over, disposed about, and/or disposed radially outward of at least a portion of the expandable frame 110 and/or the plurality of struts, as seen in FIG. 3. In some embodiments, the occlusive element 120 may be attached to the proximal hub 112 and/or may be attached to the expandable frame 110 at the proximal hub 112. In some embodiments, the occlusive element 120 may extend radially outward from and/or may extend distally from the proximal hub 112. In some embodiments, the occlusive element 120 may be attached and/or secured to the expandable frame 110 at a plurality of discrete locations. In some embodiments, one of, some of, and/or all of the at least one anchoring member 116 may extend through an occlusive element 120, where present.

In some embodiments, the occlusive element 120 may include a membrane, a fabric, a mesh, a tissue element, or another suitable construction. In some instances, the occlusive element 120 may include a fabric that has one or more coatings dispersed on or within the fabric. In some embodiments, the occlusive element 120 may be porous. In some embodiments, the occlusive element 120 may be non-porous. In some embodiments, the occlusive element 120 may be permeable to selected gases and/or fluids. In some embodiments, the occlusive element 120 may be substantially impermeable to selected gases and/or fluids, such as blood, water, etc. In some embodiments, the occlusive element 120 may be designed, sized, and/or configured to prevent thrombus and/or embolic material from passing out of the left atrial appendage into the left atrium and/or the patient's bloodstream. In some embodiments, the occlusive element 120 may be configured to promote endothelization after implantation, thereby effectively removing the target site (e.g., the left atrial appendage, etc.) from the patient's circulatory system. Some suitable, but non-limiting, examples of materials for the occlusive element 120 are discussed below.

In some cases, an LAAC device (such as but not limited to the LAAC device 100, may include additional features that are adapted to facilitate thrombus formation from a backside of the LAAC device. This can reduce blood flow through and around the LAAC device, for example.

In some cases, an LAAC device may include one or more space filling elements that are adapted to fit within an interior of the LAAC device, such that the space filling elements are within the LAA 10. In some cases, the space filling elements may be considered as being adapted to facilitate thrombus formation. In some cases, the expandable frame 100 and/or the spacing filling elements may include or be coated with a swellable material that is adapted to swell, or grow in volume, once the swellable material contacts blood. The swellable material may be a hydrogel. A hydrogel is a cross-linked hydrophilic polymer that does not dissolve in water, and can be prepared from either naturally occurring polymers or synthetic polymers. Examples of natural polymers that may be used for making a hydrogel include but are not limited to hyaluronic acid, chitosan, heparin, alginate and fibrin. Examples of synthetic polymers that may be used for making a hydrogel include but are not limited to PVA (poly vinyl alcohol), PEG (polyethylene glycol), sodium polyacrylate, acrylate polymers and copolymers thereof. The space filling elements may include one or more embolic coils, for example, that are secured to the expandable frame 110 prior to implantation.

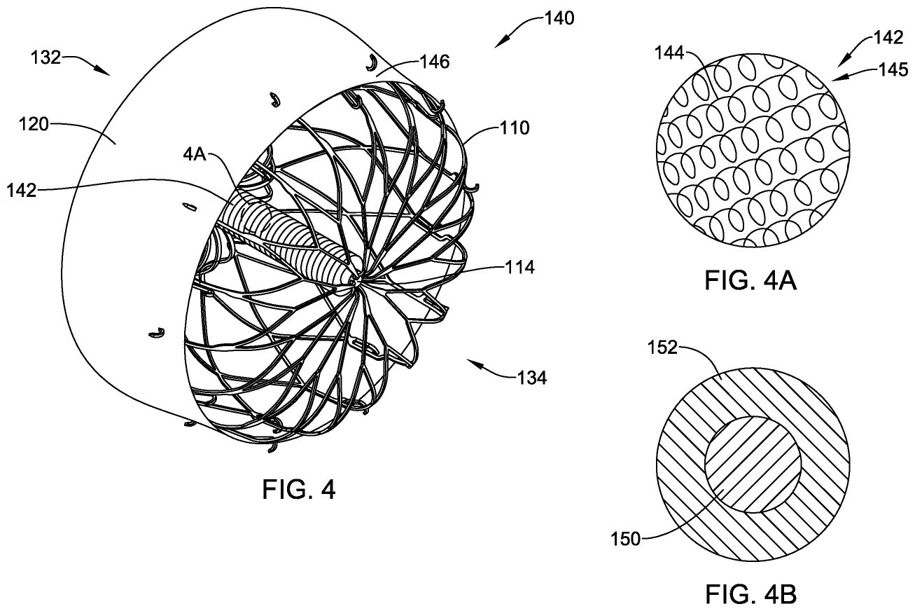

FIG. 4 is a perspective view of an illustrative LAAC device 140 that includes the expandable frame 110 and the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 140 is implanted within the LAA 10. In some embodiments, the proximal hub 112 (seen in for example in FIG. 2) and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 140 and/or the expandable frame 110 to a delivery device.

The illustrative LAAC device 140 includes a space filling element 142 that extends between the proximal hub 112 and the distal hub 114. In some cases, the space filling element 142 may include an embolic coil that may be attached at one end to either the proximal hub 112 or the distal hub 114. In some cases, the embolic coil may be attached at one end to the proximal hub 112 and at an opposing end to the distal hub 114. In some cases, the space filling element 142 may include an embolic coil such as those commercially available from Boston Scientific under the EMBOLD™ and INTERLOCK™ brand names. As shown, the space filling element 142 is shown in a contracted or collapsed configuration. It will be appreciated that the space filling element 142 may be adapted to expand into an expanded configuration in which the space filling element 142 randomly coils over itself, providing additional surface area for encouraging thrombus formation.

In some cases, the space filling element 142 may be considered as being a coiled coil such as the aforementioned EMBOLD™ and INTERLOCK™ embolic coils. FIG. 4A is an enlarged view of a portion of the space filling element 142, showing how the space filling element 142 includes one or more elongate members 144 that forms a coil 145. The coil 145 is itself coiled around a longitudinal axis to form an embolic coil such as the aforementioned EMBOLD™ and INTERLOCK™ embolic coils, serving as the space filling element 142. The space filling element may be formed of a variety of materials, including but not limited to shape memory materials that can have a remembered shape, be temporarily deformed from the remembered shape (such as for delivery) and can then regain the remembered shape when no longer constrained from doing so. Shape memory materials include shape memory metals such as Nitinol and a variety of different shape memory polymers.

In some cases, while not shown in FIG. 4 or FIG. 4A, the space filling element 142 may include one or more fibers or ribbons extending from the space filling element 142. The fibers or ribbons may provide additional surface area in order to encourage thrombus formation. The fibers or ribbons may be formed of any suitable fibrous material, such as but not limited to polymeric materials such as a polyester. This is just an example, and other materials are contemplated.

In some cases, and with reference to FIG. 4, the LAAC device 140 may be considered as including the expandable frame 110, which is moveable between a collapsed configuration for delivery and an expanded configuration for deployment. The space filling element 142 may be considered as being constrained by the expandable frame 110. The occlusive element 120 spans at least part of the expandable frame 110, and the space filling element 142 is adapted to facilitate thrombus formation. In some cases, as shown in FIG. 4, the space filling element 142 may be constrained by being secured to the expandable frame 110 prior to implantation of the LAAC device 140. As an example, the space filling element 142 may be welded to part of the expandable frame 110, such as being welded to at least one of the proximal hub 112 and the distal hub 114. As another example, the space filling element 142 may be adhesively secured to part of the expandable frame 110, such as being adhesively secured to at least one of the proximal hub 112 and the distal hub 114. Suitable adhesives include those in the cyanoacrylate family, epoxy and a variety of UV curable adhesives. The space filling element 142 may be secured in place via a mechanical interaction with the expandable frame 110. It will be appreciated that depending on the exact form of the space filling element 142, and/or how many space filling elements 142 there are, that they may be secured to the expandable frame 110 in any of a variety of locations, using a variety of attachment techniques.

In some cases, a portion or portions of the LAAC device 140 may be coated with or otherwise include materials that facilitate thrombus formation. FIG. 4B is a schematic cross-sectional view of a component 150 that includes a coating 152 that is disposed on the component 150. It will be appreciated that the component 150 may represent a strut within the expandable frame 110. In some cases, the component 150 may represent one or more of the space filling elements 142. The coating 152 may represent a swellable material such as a hydrogel. In some cases, the coating 152 may represent a material that is adapted to encourage tissue growth. The coating 152 may be applied in any suitable fashion, such as dip coating or spraying the coating 152 onto the component 150.

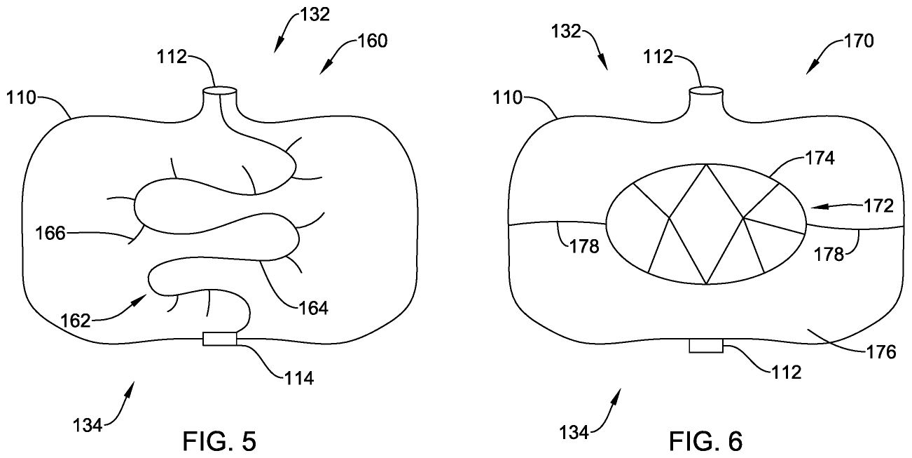

FIG. 5 is a schematic view of an illustrative LAAC device 160 that includes the expandable frame 110 including the proximal hub 112 and the distal hub 114. While not expressly illustrated, the LAAC device 160 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 160 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 160 and/or the expandable frame 110 to a delivery device.

As shown, the LAAC device 160 includes a space filling element 162 that extends from the proximal hub 112 to the distal hub 114. In some instances, the space filling element 162 is secured relative to the expandable frame 110 prior to implantation of the LAAC device 160. In some cases, the space filling element 162 is welded, soldered or adhesively secured at either end to the proximal hub 112 and the distal hub 114, for example. In some cases, the space filling element 162 includes a structural element 164 having a number of fibers 166 extending outwardly from the structural element 164. The fibers 166 may help to fill up a greater fraction of the interior volume of the LAAC 160. The fibers 166 may be tied to the structural element 164. In some cases, the fibers 166 may be adhesively secured to the structural element 164.

In some cases, at least some of the fibers 166 may be coated with, or otherwise include materials to facilitate thrombus facilitation. The structural element 164 may be considered as being an embolic coil, for example, such as those noted with respect to FIG. 4. The structural element 164 may be welded to the expandable frame 110, for example, or may be adhesively secured to the expandable frame 110. Example materials for the structural element 164 include but are not limited to Nitinol, stainless steel, titanium and other metals. Example materials for the fibers 166 include but are not limited to polyester, PEG (polyethylene glycol), PLA (polylactic acid), PLGA (poly(lactic-co-glycolic acid)), PGA (poly(glycolic acid)), PTFE (polytetrafluoroethylene) and others.

As shown, the structural element 164 follows a convoluted path between the proximal hub 112 and the distal hub 114. This has several benefits. The convoluted path means that the structural element 164 fills up a relatively larger fraction of an interior volume of the LAAC device 160. The convoluted path also means that when the LAAC device 160 is in a collapsed configuration for delivery, the structural element 164 has sufficient length to be able to span from the proximal hub 112 to the distal hub 114. While the individual fibers 166 are illustrated as being relatively short, it will be appreciated that in some cases at least some of the individual fibers 166 may be substantially longer, and thus may consume a greater fraction of the volume within the LAAC device 160.

FIG. 6 is a schematic view of an illustrative LAAC device 170 that includes the expandable frame 110 including the proximal hub 112 and the distal hub 114. While not expressly illustrated, the LAAC device 170 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 170 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 170 and/or the expandable frame 110 to a delivery device.

As shown, the LAAC device 170 includes a space filling element 172 that is disposed within an interior volume 176 of the expandable frame 110. In some instances, the space filling element 172 is captive within the expandable frame 110 but is not secured to the expandable frame 110. In some instances, the space filling element 162 is disposed captive by the expandable frame 110 prior to implantation of the LAAC device 160. In some cases, the space filling element 172 includes an expandable central element 174 that is adapted to expand to fill an interior volume 176 of the LAAC device 170. In some cases, the expandable central element 174 may be similar to the expandable frame 110 itself, but may be smaller than the expandable frame 110 such that the expandable central element 174 is able to fit within the expandable frame 110. In some cases, the expandable central element 174 may expand as the expandable frame 110 expands. In some instances, the expandable central element 174 may be formed of Nitinol. In some cases, the expandable central element 174 may be created from Nitinol coils. The expandable central element 174 may include a plurality of coils, or could be a single coil that is wound into a larger secondary coil. In some instances, the expandable central element 174 may not be self-expanding, but instead may be caused to expand when the expandable frame 110 expands. Accordingly, the expandable central element 174 may be formed of steel or another metal.

In some cases, the expandable central element 174 may include one or more ties or other elements 178 that connect the expandable central element 174 to the expandable frame 110. As the expandable frame 110 expands during deployment, the ties or other elements 178 exert a radial force on the expandable central element 174, thereby causing the expandable central element 174 to expand as well. In some cases, when the expandable central element 174 is self-expanding, the ties or other elements 178 may be excluded, or may be used only for locating the expandable central element 174 within the interior volume 176.

In some instances, the expandable central element 174 may further include fibers (not shown) that extend outwardly from the expandable central element 174. Example materials for the expandable central element 174 include but are not limited to Nitinol, stainless steel, titanium and other metals. Example materials for the fibers, if present, include but are not limited to polyester, PEG (polyethylene glycol), PLA (polylactic acid), PLGA (poly(lactic-co-glycolic acid)), PGA (poly(glycolic acid)), PTFE (polytetrafluoroethylene) and others. Portions of the LAAC device 170 may be coated with or otherwise include materials that facilitate thrombus formation.

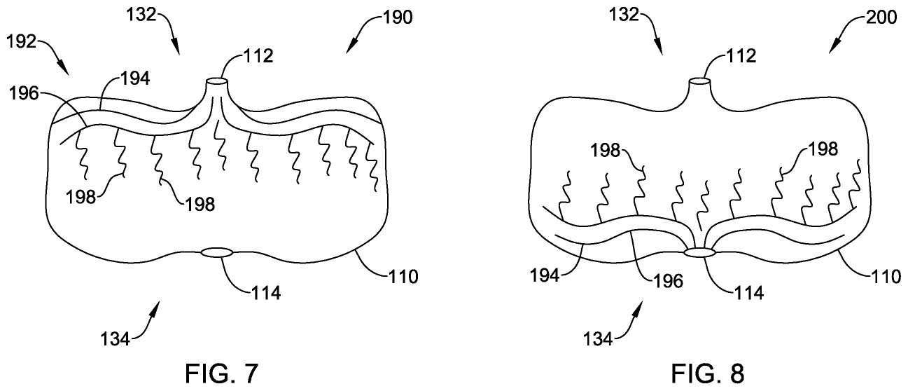

FIG. 7 is a schematic view of an illustrative LAAC device 190 that includes the expandable frame 110 including the proximal hub 112 and the distal hub 114. While not expressly illustrated, the LAAC device 190 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 190 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 190 and/or the expandable frame 110 to a delivery device.

As shown, the LAAC device 190 includes a space filling element 192 that is disposed near the proximal-facing side 132 of the expandable frame 110. The space filling element 192 includes a structural element 194 that extends radially outwardly from the proximal hub 112. The space filling element 192 includes a structural element 196 that extends radially outwardly from the proximal hub 112. The structural element 194 and the structural element 196 may each be welded or adhesively secured to the proximal hub 112. In some cases, the structural element 196 may include a number of fibers 198 that extend from the structural element 196 into an interior of the LAAC device 190. The fibers 198 may be tied or adhesively secured to the structural element 196. The space filling element 192 may be disposed within the LAAC device 190 prior to implantation.

While not shown, in some instances, the structural element 194 may also include a number of fibers 198 extending from the structural element 194. In some instances, the space filling element 192 may include the structural element 196 but not include the structural element 194. In some instances, the space filling element 192 may include additional structural elements as well. The structural elements 192 and 194 may be welded to the expandable frame 110, for example, or may be adhesively secured to the expandable frame 110.

Example materials for the structural element 194 and/or the structural element 196 include but are not limited to Nitinol, stainless steel, titanium and other metals. Example materials for the fibers 198 include but are not limited to polyester, PEG (polyethylene glycol), PLA (polylactic acid), PLGA (poly(lactic-co-glycolic acid)), PGA (poly(glycolic acid)), PTFE (polytetrafluoroethylene) and others. Portions of the LAAC device 190 may be coated with or otherwise include materials that facilitate thrombus formation.

FIG. 8 is a schematic view of an illustrative LAAC device 200 that includes the expandable frame 110 including the proximal hub 112 and the distal hub 114. While not expressly illustrated, the LAAC device 200 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 200 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 200 and/or the expandable frame 110 to a delivery device.

As shown, the LAAC device 200 includes a space filling element 200 that is disposed near the distal-facing side 134 of the expandable frame 110. The space filling element 192 includes a structural element 194 that extends radially outwardly from the distal hub 114. The space filling element 192 includes a structural element 196 that extends radially outwardly from the distal hub 114. The structural element 194 and the structural element 196 may be welded or adhesively secured to the distal hub 114. In some cases, the structural element 196 may include a number of fibers 198 that extend from the structural element 196 into an interior of the LAAC device 200. The fibers 198 may be tied or adhesively secured to the structural element 196. The space filling element 192 may be disposed within the LAAC device 190 prior to implantation.

While not shown, in some instances, the structural element 194 may also include a number of fibers 198 extending from the structural element 194. In some instances, the space filling element 192 may include the structural element 196 but not include the structural element 194. In some instances, the space filling element 192 may include additional structural elements as well. The structural elements 194 and 196 may be welded to the expandable frame 110, for example, or may be adhesively secured to the expandable frame 110.

Example materials for the structural element 194 and/or the structural element 196 include but are not limited to Nitinol, stainless steel, titanium and other metals. Example materials for the fibers 198 include but are not limited to polyester, PEG (polyethylene glycol), PLA (polylactic acid), PLGA (poly(lactic-co-glycolic acid)), PGA (poly(glycolic acid)), PTFE (polytetrafluoroethylene) and others. Portions of the LAAC device 200 may be coated with or otherwise include materials that facilitate thrombus formation.

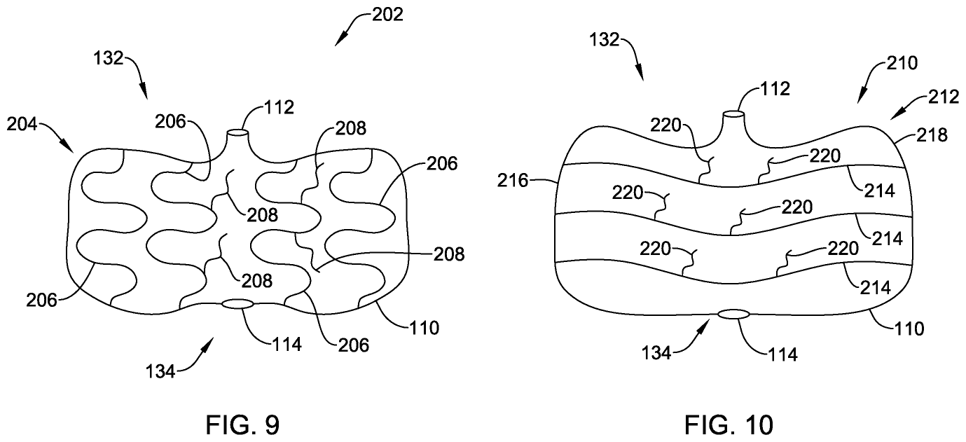

FIG. 9 is a schematic view of an illustrative LAAC device 202 that includes the expandable frame 110 including the proximal hub 112 and the distal hub 114. While not expressly illustrated, the LAAC device 202 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 202 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 202 and/or the expandable frame 110 to a delivery device.

As shown, the LAAC device 202 includes a space filling element 204 that spans the expandable frame 110. The space filling element 204 may be secured relative to the expandable frame 110 prior to implantation. The space filling element 204 includes a number of fibers 206 that extend from the proximal-facing side 132 of the expandable frame 110 to the distal-facing side 134 of the expandable frame 110. The fibers 206 may be tied to the expandable frame 110. In some cases, the fibers 206 may be adhesively secured to the expandable frame 110. In some cases, at least some of the fibers 206 follow a convoluted path between the proximal-facing side 132 of the expandable frame 110 to the distal-facing side 134 of the expandable frame 110. This has several benefits. The convoluted path means that the fibers 206 each fill up a relatively larger fraction of an interior volume of the LAAC device 202. The convoluted path also means that when the LAAC device 202 is in a collapsed configuration for delivery, the fibers 206 have sufficient length to be able to span from the proximal-facing side 132 of the expandable frame 110 to the distal-facing side 134 of the expandable frame 110. In some cases, at least some of the fibers 206 may additionally include fibers 208 extending out from the fibers 206. Example materials for the fibers 206 and 208 include but are not limited to polyester, PEG (polyethylene glycol), PLA (polylactic acid), PLGA (poly(lactic-co-glycolic acid)), PGA (poly(glycolic acid)), PTFE (polytetrafluoroethylene) and others. Portions of the LAAC device 202 may be coated with or otherwise include materials that facilitate thrombus formation.

FIG. 10 is a schematic view of an illustrative LAAC device 210 that includes the expandable frame 110 including the proximal hub 112 and the distal hub 114. While not expressly illustrated, the LAAC device 202 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 202 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 202 and/or the expandable frame 110 to a delivery device.

As shown, the LAAC device 210 includes a space filling element 212 that spans the expandable frame 110. The space filling element 212 may be secured in position prior to implantation of the LAAC device 210. The space filling element 212 includes a number of fibers 214 that extend from a first side 216 of the expandable frame 110 to a second side 218 of the expandable frame 110. While this is a two-dimensional illustration, it will be appreciated that at least some of the fibers 214 may be disposed within the plane of the paper while others of the fibers 214 may be disposed within other planes, such that some of the fibers 214 may cross or intersect others of the fibers 214 within an interior of the expandable frame 110. The fibers 214 may be tied or adhesively secured to the expandable frame 110. In some cases, at least some of the fibers 214 may additionally include fibers 220 that extend outwardly from the fibers 214.

Example materials for the fibers 214 and 220 include but are not limited to polyester, PEG (polyethylene glycol), PLA (polylactic acid), PLGA (poly(lactic-co-glycolic acid)), PGA (poly(glycolic acid)), PTFE (polytetrafluoroethylene) and others. Portions of the LAAC device 210 may be coated with or otherwise include materials that facilitate thrombus formation.

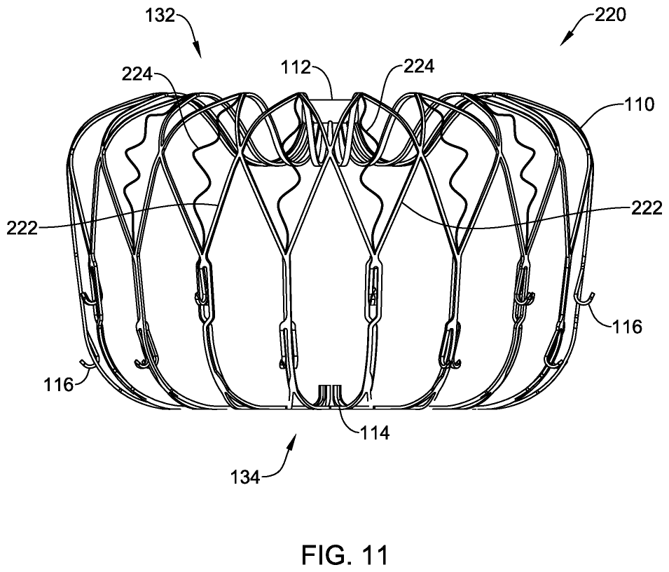

FIG. 11 is a side view of an illustrative LAAC device 220 that includes the expandable frame 110 including the proximal hub 112 and the distal hub 114. While not expressly illustrated, the LAAC device 220 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 220 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 220 and/or the expandable frame 110 to a delivery device.

The expandable frame 110 includes a number of cells 222 that are formed via periodic intersections of adjoining struts forming the expandable frame 110. The cells 222 are disposed near the proximal-facing side 132 of the expandable frame 110. In some cases, the LAAC device 220 may include a number of fibers 224 that extend within some of the cells 222. The fibers 224 may be tied to the expandable frame 110, for example. In some cases, the fibers 224 may be adhesively secured to the expandable frame 110. Example materials for the fibers 224 include but are not limited to polyester, PEG (polyethylene glycol), PLA (polylactic acid), PLGA (poly(lactic-co-glycolic acid)), PGA (poly(glycolic acid)), PTFE (polytetrafluoroethylene) and others. Portions of the LAAC device 220 may be coated with or otherwise include materials that facilitate thrombus formation.

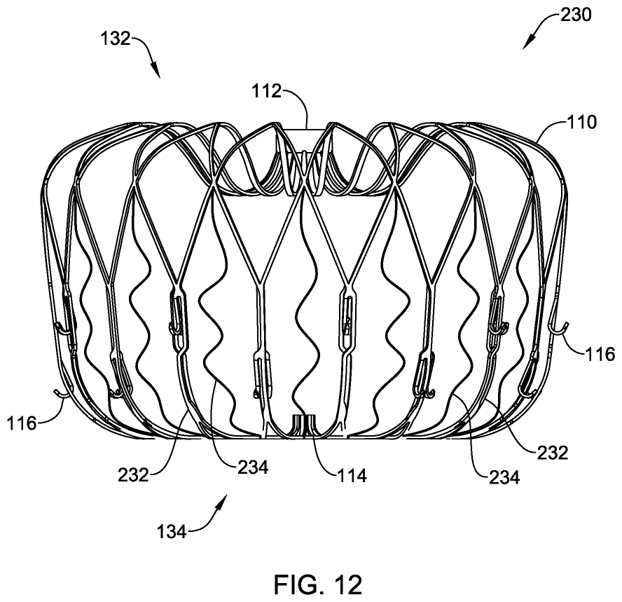

FIG. 12 is a side view of an illustrative LAAC device 230 that includes the expandable frame 110 including the proximal hub 112 and the distal hub 114. While not expressly illustrated, the LAAC device 230 may include the occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 230 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 230 and/or the expandable frame 110 to a delivery device.

The expandable frame 110 includes a number of cells 232 that are formed via periodic intersections of adjoining struts forming the expandable frame 110. The cells 232 are disposed near the distal-facing side 134 of the expandable frame 110. In some cases, the LAAC device 230 may include a number of fibers 234 that extend within some of the cells 232. The fibers 234 may be tied to the expandable frame 110, for example. In some cases, the fibers 234 may be adhesively secured to the expandable frame 110. Example materials for the fibers 234 include but are not limited to polyester, PEG (polyethylene glycol), PLA (polylactic acid), PLGA (poly(lactic-co-glycolic acid)), PGA (poly(glycolic acid), PTFE (polytetrafluoroethylene)) and others. Portions of the LAAC device 230 may be coated with or otherwise include materials that facilitate thrombus formation. While the LAAC device 220 shown in FIG. 11 shows fibers 224 located within proximal-side cells 222 and the LAAC device 230 shown in FIG. 12 shows fibers 234 located within distal-side cells 232, in some cases an LAAC device may include both the fibers 224 within proximal-side cells 222 and the fibers 234 within distal-side cells 232.

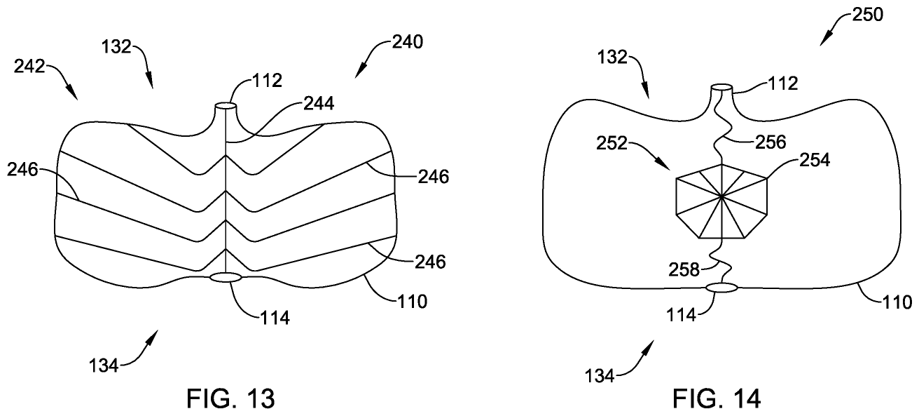

FIG. 13 is a schematic view of an illustrative LAAC device 240 that includes the expandable frame 110 including the proximal hub 112 and the distal hub 114. While not expressly illustrated, the LAAC device 240 may include occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 230 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 230 and/or the expandable frame 110 to a delivery device.

The LAAC device 240 includes a space filling element 242. In some cases, the space filling element 242 may include a central member 244 that extends from the proximal hub 112 to the distal hub 114, and may be welded, soldered or adhesively secured at either end to the proximal hub 112 and the distal hub 114. Example materials for the central member 224 include but are not limited to Nitinol, stainless steel, titanium and other metals. The space filling element 242 may also include a number of elements 246 that are secured at one end to the central member 246 and extend radially outwardly. In some cases, the elements 246 may be secured to a periphery of the expandable frame 110. In some cases, the elements 246 may be adapted to roll up around the central member 224 when the LAAC device 240 is in its collapsed configuration (not shown).

In some cases, the elements 246 may be fibers. Example materials for the elements 246 include but are not limited to polyester, PEG (polyethylene glycol), PLA (polylactic acid), PLGA (poly(lactic-co-glycolic acid)), PGA (poly(glycolic acid), PTFE (polytetrafluoroethylene)) and others. Portions of the LAAC device 220 may be coated with or otherwise include materials that facilitate thrombus formation.

FIG. 14 is a schematic view of an illustrative LAAC device 230 that includes the expandable frame 110 including the proximal hub 112 and the distal hub 114. While not expressly illustrated, the LAAC device 230 may include occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 230 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 230 and/or the expandable frame 110 to a delivery device.

As shown, the LAAC device 250 includes a space filling element 252 that extends from the proximal hub 112 to the distal hub 114. In some cases, the space filling element 252 includes an expandable central element 254 that is adapted to expand to fill an interior volume of the LAAC device 250. In some cases, the space filling element 252 is welded, soldered or adhesively secured at either end to the proximal hub 112 and the distal hub 114, for example. In some cases, the expandable central element 254 includes or is attached to a first member 256 that secures the expandable central element 254 to the proximal hub 112 and includes or is attached to a second member 258 that secures the expandable central element 254 to the distal hub 114. The first member 256 and the second member 258 may have lengths sufficient to accommodate the collapsed delivery configuration of the expandable frame 110, and thus may have an undulating configuration when the expandable frame 110 is in its expanded configuration in which the expanded frame 110 has an expanded width and a reduced length or height relative to its collapsed configuration.

The expandable central element 254 may be adapted to have a collapsed configuration for deployment, and may automatically expand into an expanded configuration when the expandable frame 110 expands, for example. In some cases, the first member 256 may extend from the proximal hub 112 to a distal side of the expandable central element 254 and the second member 258 may extend from the distal hub 114 to a proximal side of the expandable central element 254. The expandable central element 254 may expand as the expandable frame 110 moves from its collapsed configuration to its expanded configuration, for example. Example materials for the expandable central element 254, the first member 256 and the second member 258 may each include but are not limited to Nitinol, stainless steel, titanium and other metals. Portions of the LAAC device 250 may be coated with or otherwise include materials that facilitate thrombus formation.

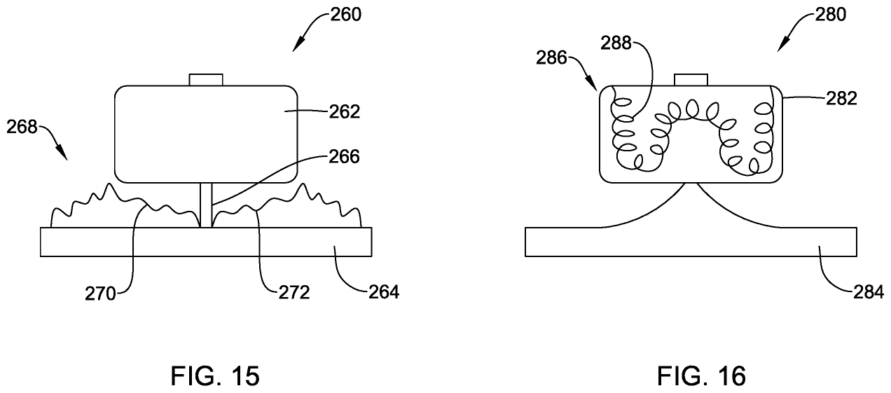

FIG. 15 is a schematic view of an illustrative LAAC device 260 that includes a first expandable feature 262 and a second expandable feature 264. Each of the first expandable features 262 and 264 may be considered as being moveable between a collapsed configuration for delivery and an expanded configuration (as shown) for deployment. The first expandable feature 262 and the second expandable feature 264 may each be laser cut structures similar to how the expandable frame 110 is formed. The first expandable feature 262 and the second expandable feature 264 may each be braided or woven structures, and may be formed of any suitable material. The LAAC device 260 includes a central member 266 that joins together the first expandable feature 262 and the second expandable feature 264. The central member 266 may be formed as a separate element, or may be part of the first expandable feature 262 and/or the second expandable feature 264, and may be formed of any suitable material.

The LAAC device 260 includes a space filling element 268 that are disposed between the first expandable feature 262 and the second expandable feature 264. In some cases, for example, the space filling element 268 may include a first element 270 that is located on a left (in the illustrated orientation) side of the central member 266 and a second element 272 that is located on a right side of the central member 266. The first element 270 and the second element 272 may each be metallic structures such as the embolic coils referenced herein. The first element 270 and the second element 272 may each be fibrous structures such as ribbons or fibers, for example.

Example materials for the first element 270 and the second element 272 include but are not limited to Nitinol, stainless steel, titanium and other metals. Example materials for the first element 270 and the second element 272 each include but are not limited to polyester, PEG (polyethylene glycol), PLA (polylactic acid), PLGA (poly(lactic-co-glycolic acid)), PGA (poly(glycolic acid), PTFE (polytetrafluoroethylene)) and others. Portions of the LAAC device 260 may be coated with or otherwise include materials that facilitate thrombus formation.

FIG. 16 is a schematic view of an illustrative LAAC device 280 that includes a first expandable feature 282 and a second expandable feature 284. Each of the first expandable features 282 and 284 may be considered as being moveable between a collapsed configuration for delivery and an expanded configuration (as shown) for deployment. The first expandable feature 282 and the second expandable feature 284 may each be laser cut structures similar to how the expandable frame 110 is formed. The first expandable feature 282 and the second expandable feature 284 may each be braided or woven structures, and may be formed of any suitable material. In some cases, as shown, the second expandable feature 284 may be adapted to be secured directly to the first expandable feature 282.

The LAAC device 280 includes a space filling element 286 that may be disposed within the first expandable feature 282, within the second expandable feature 284, or within both the first expandable feature 282 and the second expandable feature 284. As shown, the space filling element 286 is disposed within the first expandable feature 282 and includes an embolic coil 288 that is a coiled coil. In some cases, the space filling element 286 may include fibrous material such as ribbons or fibers disposed within at least one of the first expandable feature 282 and the second expandable feature 284.

Example materials for the space filling element 286 include but are not limited to Nitinol, stainless steel, titanium and other metals. Example materials for the space filling element 286 include but are not limited to polyester, PEG (polyethylene glycol), PLA (polylactic acid), PLGA (poly(lactic-co-glycolic acid)), PGA (poly(glycolic acid), PTFE (polytetrafluoroethylene)) and others. Portions of the LAAC device 280 may be coated with or otherwise include materials that facilitate thrombus formation.

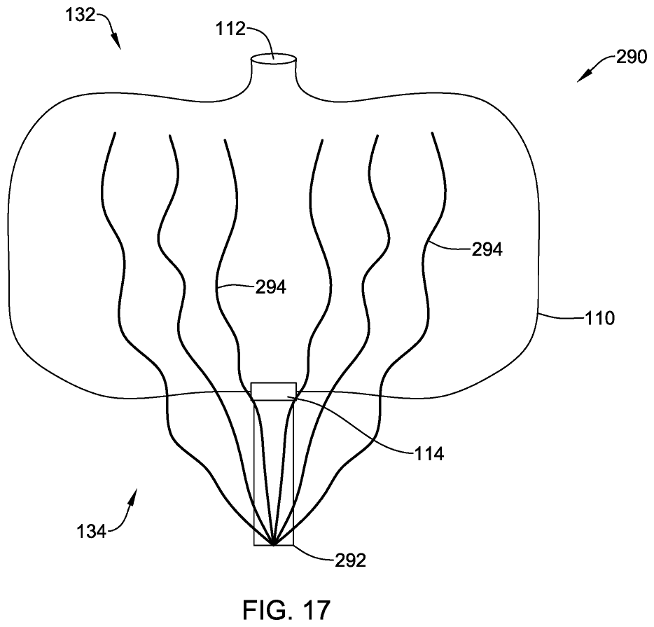

FIG. 17 is a schematic view of an illustrative LAAC device 290 that includes the expandable frame 110 including the proximal hub 112 and the distal hub 114. While not expressly illustrated, the LAAC device 240 may include occlusive element 120. The expandable frame 110 may be considered as having a proximal-facing side 132 and a distally-facing side 134. It will be appreciated that the proximal-facing side 132 will face proximally and the distal-facing side 134 will face distally when the LAAC device 230 is implanted within the LAA 10. In some embodiments, the proximal hub 112 and/or the distal hub 114 may be centered on and/or coaxial with a longitudinal axis. The plurality of struts may be joined together at and/or fixedly attached to the proximal hub 112 and/or the distal hub 114. The proximal hub 112 may be configured to releasably connect, secure, and/or attach the LAAC device 230 and/or the expandable frame 110 to a delivery device.

The LAAC device 290 includes an elongate member 292 extending distally from the distal hub 114. The elongate member 292 may be welded or adhesively secured to the distal hub 114. In some cases, the elongate member 292 may be integrally formed as part of the expandable frame 110. A plurality of fibers 294 extend from a distal end of the elongate member 292 and extend proximally into an interior of the expandable frame 110. In some instances, there may be delivery advantages in the LAAC device 290 being adapted to have the plurality of fibers 294 located outside of the expandable frame 110 for delivery. Once the LAAC device 290 is deployed, the plurality of fibers 294 are allowed to extend proximally into the expandable frame 110. Example materials for the fibers 294 include but are not limited to polyester, PEG (polyethylene glycol), PLA (polylactic acid), PLGA (poly(lactic-co-glycolic acid)), PGA (poly(glycolic acid), PTFE (polytetrafluoroethylene)) and others. Portions of the LAAC device 290 may be coated with or otherwise include materials that facilitate thrombus formation.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene

(PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device, comprising:
an expandable frame moveable between a collapsed configuration for delivery and an expanded configuration for deployment;
one or more space filling elements constrained by the expandable frame; and
an occlusive element spanning at least part of the expandable frame;
wherein the one or more space filling elements are adapted to facilitate thrombus formation within the LAAC device.

2. The LAAC device of claim 1, wherein at least some of the one or more space filling elements are constrained by being secured to the expandable frame prior to implantation of the LAAC device.

3. The LAAC device of claim 2, wherein at least some of the one or more space filling elements are welded to the expandable frame.

4. The LAAC device of claim 2, wherein at least some of the one or more space filling elements are adhesively secured to the expandable frame.

5. The LAAC device of claim 1, wherein the expandable frame further comprises a proximal hub and a distal hub, and at least some of the one or more space filling elements are secured to at least one of the proximal hub and the distal hub.

6. The LAAC device of claim 1, wherein at least some of the one or more space filling elements are constrained by being trapped within the expandable frame prior to implantation.

7. The LAAC device of claim 1, wherein at least some of the one or more space filling elements comprise an embolic coil.

8. The LAAC device of claim 1, wherein at least some of the one or more space filling elements comprises fibrous material.

9. The LAAC device of claim 1, wherein the expandable frame comprises a plurality of struts forming a plurality of strut intersections, and at least some of the one or more space filling elements comprise fibers each extending between two or more of the plurality of struts and/or two or more of the plurality of strut intersections.

10. The LAAC device of claim 1, wherein at least one of the one or more space filling elements comprise:
an expandable element that is adapted to be secured to the expandable frame; and
one or more fibers extending from the expandable element.

11. The LAAC device of claim 1, wherein the expandable frame comprises:
a first expandable feature that is moveable between a collapsed configuration for delivery and an expanded configuration for deployment; and
a second expandable feature that is secured relative to the first expandable feature and that is moveable between a collapsed configuration for delivery and an expanded configuration for deployment.

12. The LAAC device of claim 11, wherein at least some of the one or more space filling elements extend between the first expandable feature and the second expandable feature.

13. The LAAC device of claim 11, wherein at least some of the one or more space filling elements are disposed within at least one of the first expandable feature and the second expandable feature.

14. The LAAC device of claim 1, further comprising a coating disposed over at least a portion of the LAAC device, the coating adapted to encourage thrombus formation.

15. A left atrial appendage closure (LAAC) device, comprising:
an expandable frame moveable between a collapsed configuration for delivery and an expanded configuration for deployment;
one or more expandable elements that are secured to the expandable frame and are adapted to facilitate thrombus formation within the LAAC device; and
an occlusive element spanning at least part of the expandable frame.

16. The LAAC device of claim 15, wherein at least some of the one or more expandable elements comprise an embolic coil.

17. The LAAC device of claim 15, wherein at least some of the one or more expandable elements are secured to the expandable frame prior to implantation.

18. The LAAC device of claim 15, wherein at least some of the one or more expandable elements are adapted to move between a collapsed configuration for delivery and an expanded configuration for deployment.

19. A left atrial appendage closure (LAAC) device, comprising:
an expandable frame moveable between a collapsed configuration for delivery and an expanded configuration for deployment;
one or more fibers secured relative to the expandable frame, the one or more fibers adapted to facilitate thrombus formation within the LAAC device; and
an occlusive element spanning at least part of the expandable frame.

20. The LAAC device of claim 19, further comprising a support element extending to a position exterior of the expandable frame, and the one or more fibers are secured to the support element at the position exterior of the expandable frame and extend back into the expandable frame.
